(19) United States
(12) Patent Application Publication
(10) Pub. No.: US 2021/0038160 A1
(43) Pub. Date: Feb. 11, 2021

(54) METHOD FOR DETERMINING AN ITEM OF PATIENT INFORMATION, MEDICAL ENGINEERING ARRANGEMENT, COMPUTER PROGRAM AND ELECTRONICALLY READABLE DATA CARRIER

(71) Applicant: Siemens Healthcare GmbH, Erlangen (DE)

(72) Inventor: Philip Mewes, Nürnberg (DE)

(21) Appl. No.: 16/985,210

(22) Filed: Aug. 4, 2020

(30) Foreign Application Priority Data
Aug. 5, 2019 (DE) 102019211705.8

(51) Int. Cl. A61B 5/00; A61B 5/11

(52) U.S. Cl. CPC A61B 5/70; A61B 5/1127; A61B 5/0022

(57) ABSTRACT

A method for determining an item of patient information that alludes to a position and/or a movement of a patient during an examination and/or treatment process with a medical engineering apparatus is provided. The patient information is determined from sensor data of a mobile telephone sensor of a mobile telephone, such as a smartphone, coupled motionally to the patient, at least in one direction of movement.

This application claims the benefit of German Patent Application No. DE 10 2019 211 705.8, filed on Aug. 5, 2019, which is hereby incorporated by reference in its entirety.

### BACKGROUND

The present embodiments relate to determining an item of patient information, a medical engineering arrangement, a computer program, and an electronically readable data carrier.

Over the last few years, medical engineering has made extremely significant progress, particularly with regards to examinations and treatment/therapeutic measures. One factor that is to be taken into account and is difficult to model when new engineering concepts are applied is the patient. It is therefore extremely important to monitor the patient as closely as possible during examination and/or treatment processes on the patient; this is also frequently referred to as patient monitoring. By way of example, vital parameters and other patient parameters may be recorded during an examination and/or treatment and are then used as input data for medical engineering apparatuses (e.g., therapeutic and diagnostic devices). This type of patient information is often only accessible using special monitoring apparatuses (e.g., in association with anesthesia equipment) or even not available in a proprietary manner.

The correct positioning and in this context also potential movement of the patient represents an important aspect of patient monitoring, particularly with imaging diagnostics and/or targeted treatment of specific points in the human body. In order to record patient information, which describes the position and/or movement of a patient during an examination and/or treatment process, various complex monitoring systems have already been proposed. For example, patient movements may be traced using additional optical cameras that are directed at the patient, and/or special markers (e.g., for electromagnetic positioning systems) using sensor data evaluation of special monitoring devices. It was also proposed, however, with imaging diagnostic devices (e.g., imaging apparatuses) to evaluate the image data or special measuring processes in order to obtain patient information that describes the position and/or movement of the patient.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a cost-effective alternative option to patient monitoring that still provides high-precision information, such as with respect to movement and a position of a patient, is provided.

With an embodiment of a method for determining an item of patient information, which describes the position and/or movement of a patient during an examination and/or treatment process with a medical engineering apparatus, the patient information is determined from sensor data of a mobile telephone sensor of a mobile telephone (e.g., smartphone) coupled motionally to the patient at least in one direction of movement.

The knowledge underlying the present embodiments is that mobile telephone sensors in modern mobile telephones (e.g., acceleration sensors), cameras, and suchlike have such a good quality that the mobile telephone sensors may be used in the medical engineering context.

The quality of the sensor data is sufficient to obtain suitable patient information, which describes the position and/or movement of a patient during the examination and/or treatment process, for the requirements of an examination and/or treatment process. There is further insight into mobile phones forming part of most people's personal possessions, so that the corresponding devices are available and need not be provided, especially on the medical engineering apparatus. For example, a specific position or movement-sensitive examination and/or treatment processes may be realized on medical engineering apparatuses without having to install a complicated additional system (e.g., with additional devices).

The present embodiments therefore offer a very favorable alternative in order to obtain patient information during an examination and/or treatment process (e.g., an operation and/or an imaging measure). There is no need for complex patient monitoring systems/monitoring devices. The patient information need not only be provided to the medical engineering apparatus, but instead largely also in a larger, superimposed overall system (e.g., teamplay receiver).

The present embodiments likewise benefit from modern mobile telephones (e.g., smartphones) also having communication apparatuses so that a communication link may be set up between the medical engineering apparatus and the mobile telephone. In this way, a medical engineering arrangement is finally produced, in which the control apparatus is produced by a control unit of the mobile telephone and a control unit of the medical engineering apparatus, which are connected by way of the communication link, for example. Communication links (e.g., communication links of a wireless type) may be established using WLAN, Bluetooth, or suchlike, for example.

In one such medical engineering arrangement, the sensor data may be evaluated at least partially by a control unit of the mobile telephone. This provides that sensor data that is already evaluated or preevaluated is at least partially transmitted via the communication link as evaluation information, or when the mobile telephone carries out a comprehensive evaluation, patient information may also be transmitted. In general terms, the evaluation of the sensor data may take place at least partially by the control unit of the mobile telephone and/or the control unit of the medical engineering apparatus, where the control units may form in total an overall control apparatus of the described medical engineering arrangement.

In one embodiment, because patient information may be transmitted, the communication link between the mobile telephone and the medical engineering apparatus may be at least partially encrypted so that interception of the patient information and other transmitted data is avoided. If the mobile telephone is to be embedded entirely in a higher order examination and/or treatment system, safety measures on the part of the mobile telephone may also be provided in order to avoid unauthorized access to data and information. All of this is, however, largely known within the prior art and does not, therefore, need to be shown in more detail.

Parts of the method of the present embodiments that are to be carried out by the mobile telephone may be implemented by a special computer program or a part of a computer program, which realizes the method, for mobile telephones (e.g., in the case of smartphones by an “App” (short for application or application program)). Controlled by the mobile telephone-side part of the computer program, the communication link may be established (e.g., the sensor data may be recorded, possibly at least partially evaluated, and the communication traffic may be controlled with the medical engineering apparatus).

An acceleration sensor of the mobile telephone and/or at least one gyrosensor and/or at least one camera of the mobile telephone may be used as a mobile telephone sensor. Many modern mobile telephones (e.g., smartphones) have acceleration sensors (e.g., an inertial platform) and/or gyrosensors, via which accelerations and rotations of the mobile telephone may be measured in every direction. For example, the direction in which the earth acceleration acts may also be determined. These acceleration sensors and/or gyrosensors measure with high accuracy so that, for example, over short periods of time, high-precision position and/or movement information with respect to a patient coupled motionally with the mobile telephone may also be recorded. Modern cameras in mobile telephones (e.g., smartphones) also offer an extremely high resolution, where if necessary, the mobile telephone already provides image processing possibilities that may likewise be used in order to perform parts of the evaluation of the sensor data to obtain the patient information.

When a plurality of mobile telephone sensors based on different measuring principles are used, the respective sensor data may be used in addition and/or so as to verify plausibility. For example, intermediate and/or end results (e.g., the intensity and/or direction of an occurred movement) may be determined from sensor data of acceleration sensors and sensor data of a camera in each case, and verification of plausibility takes place by a corresponding comparison. In one embodiment, the sensor data may be merged in the evaluation in order to enable a more accurate determination, with redundantly measured information, and additionally to introduce this only with information obtained with a measuring principle.

The sensor data may be determined in a mobile telephone coordinate system that is or will be registered with a patient coordinate system of the medical engineering apparatus. Already known registration methods may basically be used in order to use patient information, obtained from sensor data, that describes the positions and/or movements meaningfully in a patient coordinate system used by the medical engineering apparatus.

The registration may, for example, take place by using a registration image and/or a navigation system recorded by the medical engineering apparatus embodied as an imaging apparatus and indicating the mobile telephone and/or a marker attached thereto (e.g., with at least one marker for the navigation system arranged on the mobile telephone and/or by sensor data of one or the acceleration sensor of the mobile telephone indicating the direction of gravity). With medical engineering apparatuses configured as imaging apparatuses, it is possible, for example, to make use of the fact that the mobile telephone is visible in the image data of the imaging apparatus. A corresponding, basically known registration method may then be used in this respect.

Similarly, markers suited to the imaging modality may be arranged on the mobile telephone. Another possibility is that a navigation system (e.g., an electromagnetic position determination system) is already used (e.g., in order to trace medical devices or suchlike, such as catheters). The mobile telephone may also be embedded in a navigation system of this type. Optical cameras may optionally also be used to monitor the examination and/or treatment area. The mobile telephone may likewise be detected in the image data of the optical cameras, and the image data may be registered with the patient coordinate system. In instances in which unwanted patient movements and/or changes in position are generally to be monitored, it may already also be sufficient to identify the direction of the gravity, since an adequate reference may then be produced. This may be determined itself in most cases, however, by the mobile telephone sensors (e.g., acceleration sensors) and may serve as reference.

In specific embodiments, the patient information may be evaluated for movement monitoring of the patient. This provides that the patient (and thus the mobile telephone coupled motionally thereto) are monitored for changes in position. This may be provided if, with imaging processes as an examination process, specific areas are to remain in an image section and/or the present embodiments are applied, for example, in radiation processes, in which the beams are to targetedly strike a specific target. A movement monitoring of the patient may also be provided with minimally invasive interventions or suchlike.

In this context, the patient information may be used to verify the plausibility of an item of movement monitoring information determined by a movement monitoring apparatus of the medical engineering apparatus. In this case, the mobile telephone is finally used as a further additional apparatus for verifying the plausibility and thus further increasing the safety during the examination and/or treatment process.

Within the scope of the present embodiments, the patient information may be used for the qualitative or quantitative movement correction of an occurred movement described by the patient information and/or to output a notification with a movement of the patient described by the patient information and fulfilling an output criteria. Corresponding measures are already known from the movement monitoring systems/movement monitoring apparatuses or specific imaging and intervention applications described in the introduction. The present embodiments allow an extremely reliable, excellently available, and easily embeddable source to be used for suitable patient information (e.g., a mobile telephone, such as a smartphone). With excessive movements, as a measure, the examination and/or treatment operation of the medical engineering apparatus may be interrupted.

When an acceleration sensor and/or a gyrosensor are used, the patient information describing a patient movement may be determined by temporal integration of the sensor data. If acceleration values are integrated over time, a movement path, with a corresponding directional resolution naturally also direction-related, may be determined therefrom, as known from other applications. If a camera is used as a mobile telephone sensor, suitable image processing algorithms may be used (e.g., image processing algorithms that use the optical flow and/or algorithms in the area “structure from motion”).

In other exemplary embodiments, the patient information may also be evaluated to determine the position of the patient, where the movement monitoring of the patient will represent the main area of application.

In a development of the present embodiments, provision may be made for the mobile telephone to be motionally coupled to the patient by a mechanical coupling apparatus (e.g., so that the non-sterile mobile telephone remains at a minimum distance from the patient). If keeping the mobile telephone sterile has proven rather difficult, the mobile telephone may be arrangeable in a non-sterile area. If a movement coupling still takes place in the sterile area, mechanical coupling apparatuses may be used in order to be able to arrange the mobile telephone at a distance from the patient or at least a sterile area, but may establish movements there. In this context, provision may be made, for example, for the mechanical coupling apparatus to couple to a medical instrument inserted into the patient and/or to include the same. This may be provided, for example, with interventions on the spinal column, where identifying the smallest of movements may be important. For example, the mechanical coupling apparatus may form an extension of the medical instrument from the sterile area.

In this context, already sterilizable types of mobile telephones have also been provided, just like the use of suitable sterilizable sheaths, so that the use of mobile telephones is at least basically conceivable also in sterile areas.

The mobile telephone may also be used at least partially as a control apparatus for the medical engineering apparatus. For example, it was already proposed in the prior art to use mobile devices with a touchscreen, of which, for example, a smartphone also forms part, as a control apparatus for medical engineering apparatuses (e.g., imaging apparatuses) to, for example, avoid interfering control consoles that may only be sterilized with difficulty.

Accordingly, a part of a computer program provided on a control unit of the mobile telephone, which realizes the method of the present embodiments, may also include parts that enable the use of the mobile telephone as a control apparatus for the medical engineering apparatus, since a communication link with the medical engineering apparatus already exists.

In a development of the present embodiments, provision may be made for at least one vital parameter of the patient also to be determined from sensor data of the or at least one further mobile telephone sensor as part of the patient information. In this case, multiple usage of the mobile telephone is provided (e.g., not only with respect to the patient monitoring, which relates to the movement and/or position, but instead, also in order to determine further patient information that includes the vital parameters). Acceleration sensors may, for example, be used within mobile telephones in order to monitor the heart beat and/or breathing of the patient. This is also possible by way of a touchscreen of the mobile telephone. A further option of determining vital parameters of a patient via a mobile telephone is provided by the use of a camera of the mobile telephone and a bright light of the mobile telephone, since this then allows the oxygen saturation to be determined. This was already proposed, for example, with special health telephones. The idea is that the mobile telephone illuminates the skin and measures the level of ultraviolet light that is absorbed. The percentage of oxygen-carrying red blood cells may be read herefrom, and the oxygen saturation may therefore be measured automatically with a mobile telephone.

In this way, therefore, both the patient information describing the movement and/or position of the patient and also patient information describing the vital parameters are determined; a large part of the required parameters are detected by a widely available, favorable mobile telephone. As a result of this, further devices (e.g., pulse meters and/or breathing belts) may be omitted. For example, it is possible to perform triggered x-ray recordings in an x-ray apparatus as a medical engineering apparatus with vital parameters supplying breathing information.

Within the scope of the present embodiments, it may be possible, for example, for the mobile telephone (e.g., using the “App”) to register with the medical engineering apparatus for the preferred examination and/or treatment process (e.g., therefore the communication link). The mobile telephone may then be placed on the patient, for example, and/or motionally coupled in other ways to the patient. The mobile telephone provides already preselected sensor data and/or as a complete evaluation result, patient information about the communication link to the medical engineering apparatus. This thus enables a monitoring of the patient at least with respect to the movement and/or position, and also with respect to vital parameters (e.g., pulse parameters, breathing parameters, and/or oxygen saturation). The patient information may be used accordingly by the medical engineering apparatus in order to take action when specific measure criteria has been fulfilled and such like.

Within the scope of the present embodiments, the patient information may be used to control the medical engineering apparatus. In addition to examples that have already been mentioned, patient information describing the position and/or the movement of a patient may be used to automatically stabilize medical instruments (e.g., catheters) controlled by robots.

With radiation applications, the patient position may be monitored continuously in order to be able to irradiate the radiation target as accurately as possible. The patient information obtained by mobile telephone sensors of a mobile telephone coupled motionally to the patient may have been used in various areas of application known essentially in the prior art, for which previously special patient monitoring apparatuses/monitoring devices have been used.

Aside from the method, the present embodiments also relate to a medical engineering arrangement including a medical engineering apparatus, a mobile telephone with at least one mobile telephone sensor, and a control apparatus (e.g., a controller) that is configured to carry out the method of one or more of the present embodiments. The control apparatus includes at least one processor and at least one storage device. The control apparatus may include the control unit of the mobile telephone and the control unit of the medical engineering apparatus. A communication link (e.g., a wireless communication link) may be set up between the control units, as described. The control apparatus may have a sensor data recording unit, for example, for collecting the sensor data from the mobile telephone sensors and an evaluation unit for evaluating sensor data, where the evaluation unit may also be provided distributed between the control unit of the mobile telephone and the control unit of the medical engineering apparatus. Both control units may have communication units in order to establish the communication link. The control unit of the medical engineering apparatus may have a measure unit, as a further unit, which evaluates the patient information in order to control the medical engineering apparatus.

A computer program has program means that execute the act of a method of the present embodiments if the computer program is executed on a control apparatus of a medical engineering arrangement, on which the computer program is loaded. The computer program may be stored on an electronically readable data carrier (e.g., a non-transitory computer-readable storage medium) that has control information stored thereupon. When the data carrier is used in a control apparatus of a medical engineering arrangement, the control information (e.g., the computer program) carries out an embodiment of the method. The data carrier may be, for example, a non-transient data carrier (e.g., a CD-ROM).

For example, the computer program may have computer program means for a mobile telephone (e.g., a smartphone) with a mobile telephone senor, which, when executed on the mobile telephone, triggers the mobile telephone to carry out the following acts: set up a communication link to form a medical engineering apparatus; receive sensor data of a mobile telephone sensor that describes a position and/or movement of the mobile telephone; and send the sensor data and/or an item of evaluation information, determined by an at least partial evaluation of the sensor data, via the communication link to the medical engineering apparatus.

All embodiments with respect to the method may be applied analogously to the medical engineering arrangement, the computer program, and the electronically readable data carrier, and vice versa. Accordingly the cited advantages may be obtained with all of these.

### BRIEF DESCRIPTION OF THE DRAWINGS

FIG. 1 is a flow chart of an exemplary embodiment of a method;

FIG. 2 shows an exemplary application in an irradiation and/or imaging process;

FIG. 3 shows an embodiment of a coupling apparatus; and

FIG. 4 is a functional drawing of one embodiment of a medical engineering arrangement.

### DETAILED DESCRIPTION

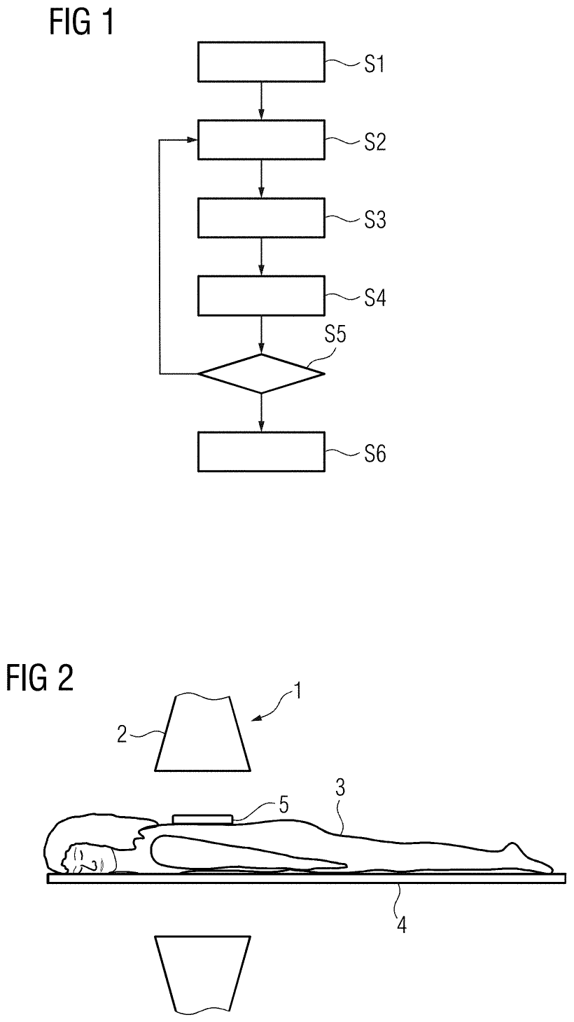

FIG. 1 shows a flow chart of an exemplary embodiment of a method. The method is started in act S1 (e.g., by selecting a corresponding function in an “App” that is installed in a mobile telephone, such as a smartphone).

This results, in act S2, in the mobile telephone setting up a wireless communication link to a medical engineering apparatus, with which an examination and/or treatment process of a patient is to be carried out. The wireless communication link may be established by WLAN and/or Bluetooth, for example. Successfully establishing the communication link, which may also include an authentication process, is indicated to the user who positions the mobile telephone in a motionally coupled manner with the patient, as explained in more detail below.

Similarly, in act S2, in this exemplary embodiment, a registration is established between a mobile telephone coordinate system of the mobile telephone and a patient coordinate system of the medical engineering apparatus.

Different registration approaches may be selected (e.g., the use of markers, the visibility of the mobile telephone in images, and/or navigation data of a navigation system or also information from mobile telephone sensors).

Accordingly, in act S3, mobile telephone sensors (e.g., acceleration sensors and/or a camera of the mobile telephone) may be used in order to record sensor data, from which patient information relating to the position and/or movement of the patient may be derived, which is provided on account of the movement coupling.

In act S4, the sensor data is evaluated in order to determine patient information that describes the movement and/or position of the patient. For example, the movement of the patient and thus an item of patient information describing a change in position may be determined by integrating sensor data of the acceleration sensors of the mobile telephone. With a camera, “structure from motion” algorithms and/or algorithms of the optical flow may be used, for example, in order to obtain corresponding information.

Sensor data from different mobile telephone sensors may be used in order to enable a mutual plausibility check; the sensor data may be merged together in order to determine the patient information. With respect to positions and orientations, the registration is used between the mobile telephone coordinate system and the patient coordinate system of the medical engineering apparatus, which was established in act S2.

In act S5, the patient information is evaluated by measure criteria (e.g., in a control unit of the medical engineering apparatus) in order to establish the need for measures within the scope of a position and/or movement monitoring of the patient. If the patient moves too much during an irradiation process, for example, the irradiation focus may be traced accordingly or with no longer correctable movements, a corresponding warning message may be output, and the irradiation may be interrupted. Movement information describing movements of the patient may also be used to stabilize robot-controlled medical instruments, which are to be used in or on the patient.

If a measure criterion is fulfilled, the corresponding measure is carried out in act S6.

In an extension of the exemplary embodiment, the recorded sensor data may be extended in act S3; an extended item of patient information is determined in act S4. This then also describes vital parameters of the patient. For example, sensor data of the acceleration sensors of the mobile telephone, which is motionally coupled to the patient, may be evaluated with respect to the pulse beat and the breathing activity, in order to be able to determine pulse rates, breathing ranges, and suchlike, for example.

In association with a bright lighting apparatus of the mobile telephone, which is typically used as a flashlight or pocket torch, the camera may be used to determine an oxygen saturation. This further patient information is also supplied via the communication link to the medical engineering apparatus and evaluated there accordingly in act S5.

Within the scope of these exemplary embodiments, the “App” on the mobile telephone may also be embodied so that the communication link is used in order to enable an at least partial operation of the medical engineering apparatus by the mobile telephone, and thus, the mobile telephone may be used as a control apparatus for the medical engineering apparatus.

FIG. 2 shows the application of the method in a medical engineering apparatus 1, which may be embodied as a combined irradiation and x-ray apparatus. The medical engineering apparatus 1 therefore has an irradiation arrangement 2 that may be positioned with respect to the patient 3 and may also be rotated about the patient 3.

The patient 3 may be supported on, for example, a patient couch 4. The mobile telephone 5 (e.g., a smartphone) may thus be positionable on the patient, for example, in order to enable a movement monitoring. The registration of the mobile telephone coordinate system with the patient coordinate system may be achieved, for example, by the visibility of the mobile telephone in x-ray images.

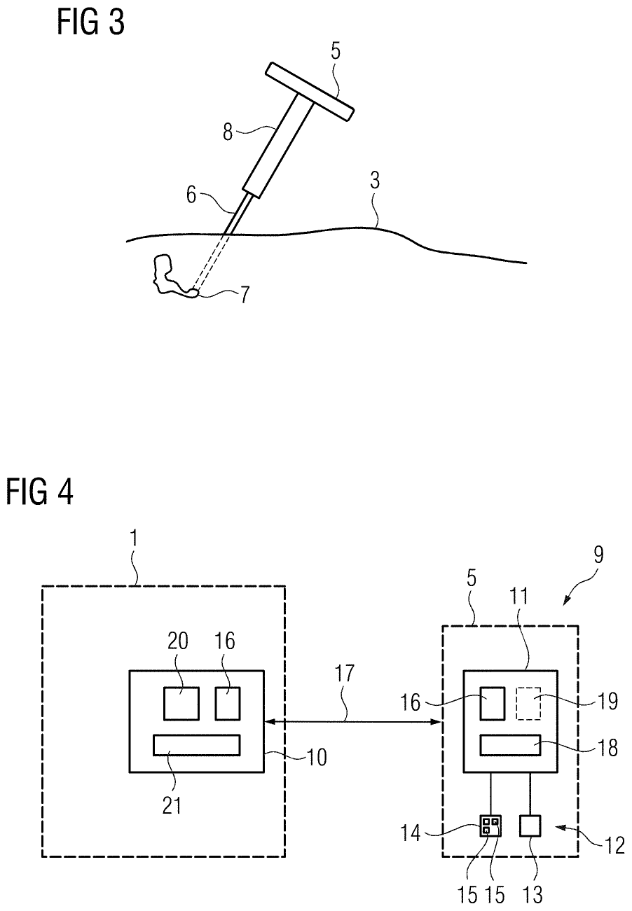

FIG. 3 shows, in a schematic view, the movement coupling of a non-sterile mobile telephone 5 to a patient 3. This is shown in the context of a spinal intervention, in which patient information describing the position and/or movement of the patient 3 is particularly useful. There, a clamping facility 6, as a medical instrument, may be fastened to a spinous process 7 inside the patient 3, where, via a further mechanical coupling apparatus 8, the mobile telephone 5 may be motionally coupled hereto at an adequate distance from the patient 3.

FIG. 4 shows the general functional design of one embodiment of a medical engineering arrangement 9. This has, as already shown, the medical engineering apparatus 1 and the mobile telephone 5, which both have a control unit 10, 11 in each case. A camera 13 and an intertial platform 14 may be provided with acceleration sensors 15 as mobile telephone sensors 12.

The control units 10, 11 (e.g., controllers) each include a communication unit 16 in order to be able to establish the communication link 17 in act S2. With respect to act S2, for the sake of clarity registration units, not shown in more detail, may also be available to establish the registration.

Sensor data is received in the control unit 11 by a sensor data recording unit 18. The sensor data may be evaluated at least partially already with respect to the patient information by an optional evaluation unit 19 of the control unit 11. If the patient information is not already determined entirely by the optional evaluation unit 19, corresponding evaluation information may arise. The corresponding evaluation information is then used by the control unit 10 (e.g., the evaluation unit 20) to determine the patient information. If there is no evaluation unit 19 in the control unit 11, the sensor data is completely evaluated in the evaluation unit 20 of the control unit 10.

A measure unit 21, in which the measure criteria for evaluating the patient information is checked and, possibly, measures are introduced, is also shown as part of the control unit 10.

This provides, while the sensor data recording unit 18 is assigned to act S3 in FIG. 1, that the evaluation units 19, 20 are to be assigned to act S4 and the measure unit 21 is to be assigned to acts S5 and S6.

Although the invention has been illustrated and described in greater detail with reference to exemplary embodiments, the invention is not restricted by the examples disclosed; other variations may be derived therefrom by the person skilled in the art without departing from the protective scope of the invention.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for determining an item of patient information that alludes to a position, movement, or position and movement of a patient during an examination, treatment, or examination and treatment process with a medical engineering apparatus, the method comprising:
determining the patient information from sensor data of a mobile telephone sensor of a mobile telephone coupled motionally to the patient at least in one direction of movement.

2. The method of claim 1, wherein the mobile telephone is a smartphone.

3. The method of claim 1, wherein at least one acceleration sensor of the mobile telephone, at least one camera of the mobile telephone, or a combination thereof is used as the mobile telephone sensor.

4. The method of claim 1, wherein when one or more other mobile telephone sensors based on different measuring principles are used, the respective sensor data of the one or more other mobile telephone sensors are used in addition, to verify plausibility, or a combination thereof.

5. The method of claim 3, wherein the sensor data is determined in a mobile telephone coordinate system that is or will be registered with a patient coordinate system of the medical engineering apparatus.

6. The method of claim 5, further comprising registering the mobile telephone coordinate system with the patient coordinate system, the registering comprising:
registering the mobile telephone coordinate system with the patient coordinate system using a registration image recorded by the medical engineering apparatus configured as an imaging apparatus, a navigation system, or a combination thereof; and
indicating the mobile telephone, a marker attached to the mobile telephone, or a combination thereof with at least one marker for the navigation system arranged on the mobile telephone, by sensor data of the at least one acceleration sensor of the mobile telephone indicating a direction of the force of gravity, or a combination thereof.

7. The method of claim 1, further comprising monitoring the movement of the patient, monitoring the movement of the patient comprising evaluating the patient information.

8. The method of claim 7, further comprising using the patient information, verifying plausibility of an item of movement monitoring information determined by a movement monitoring apparatus of the medical engineering apparatus, performing a qualitative or quantitative movement correction of an occurred movement described by the patient information, outputting a notification with a movement of the patient described by the patient information and fulfilling an output criterion, or any combination thereof.

9. The method of claim 7, wherein the patient information describing a patient movement is determined by temporal integration of the sensor data when an acceleration sensor is used.

10. The method of claim 1, wherein the mobile telephone is motionally coupled by a mechanical coupling apparatus to the patient.

11. The method of claim 10, wherein the mobile telephone is motionally coupled by the mechanical coupling apparatus to the patient, so that a non-sterile mobile telephone remains at a minimal distance from the patient.

12. The method of claim 1, wherein the mobile telephone is also used at least partially as an operator apparatus for the medical engineering apparatus.

13. The method of claim 1, further comprising determining at least one vital parameter of the patient from the sensor data of the mobile telephone sensor or sensor data of at least one further mobile telephone sensor as part of the patient information.

14. The method of claim 1, wherein the patient information is used to control the medical engineering apparatus.

15. A medical engineering arrangement comprising:
a medical engineering apparatus;
a mobile telephone including at least one mobile telephone sensor and a controller configured to determine an item of patient information that alludes to a position, movement, or position and movement of a patient during an examination, treatment, or examination and treatment process with the medical engineering apparatus, the determination of the item of patient information comprising:
determination of the patient information from sensor data of the at least one mobile telephone sensor of the mobile telephone, which is coupled motionally to the patient at least in one direction of movement.

16. In a non-transitory computer-readable storage medium that stores instructions executable by one or more processors to determine an item of patient information that alludes to a position, movement, or position and movement of a patient during an examination, treatment, or examination and treatment process with a medical engineering apparatus, the instructions comprising:
determining the patient information from sensor data of a mobile telephone sensor of a mobile telephone coupled motionally to the patient at least in one direction of movement.
